(19) United States
(12) Patent Application Publication
(10) Pub. No.: US 2010/0074854 A1
(43) Pub. Date: Mar. 25, 2010

(54) NAIL VARNISH COMPRISING A PHENYLATED SILICONE RESIN

(75) Inventors: Laurence Guerchet, Chevilly-Larue (FR); Lizabeth-Anne Coffey-Dawe, Aulnay Sous Bois (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, PARIS (FR)

(21) Appl. No.: 12/522,264

(22) PCT Filed: Jun. 4, 2008
(86) PCT No.: PCT/EP08/56917

§ 371 Date: Aug. 14, 2009

Related U.S. Application Data
(60) Provisional application No. 60/944,800, filed on Jun. 19, 2007.

(30) Foreign Application Priority Data
Jun. 6, 2007 (FR) 0755507

(51) Int. Cl. A61K 8/84; A61Q 3/02

(52) U.S. Cl. 424/61

(57) ABSTRACT

A subject-matter of the present invention is a nail varnish comprising an organic solvent medium and at least one phenylated silicone resin. Another subject-matter of the present invention is a cosmetic method for making up or for the nontherapeutic care of the nails, comprising the application, to the nails, of at least one layer of the said nail varnish, and also its use for obtaining, after deposition on the nail, a film which is glossy.

A subject-matter of the present invention is a nail varnish composition comprising an organic solvent medium and at least one phenylated silicone resin. This composition can be applied to human nails or else to false nails.

The coloured or transparent nail varnish composition can be employed as varnish base or base coat, as product for making up the nails, as finishing composition, also known as top coat, to be applied to the product for making up the nails, or else as product for the cosmetic care of the nails.

It is commonplace to use, in nail varnish compositions, film-forming agents in order to obtain, after deposition on the nail, a resistant film exhibiting good hold. Currently, nitrocellulose still remains the commonest film-forming agent used in nail varnishes exhibiting optimized properties of hold. However, these nail varnishes comprising nitrocellulose exhibit the disadvantage of not being stable towards heat and on storage: this results in yellowing of these compositions over time.

Furthermore, the introduction of large amounts of nitrocellulose conditions the composition of the organic solvent phase of the nail varnish, which then has to be compatible with this film-forming agent.

The aim of the present invention is thus to make available nail varnish compositions comprising a reduced content of nitrocellulose, indeed even devoid of nitrocellulose, which exhibit good stability over time and which make possible the formation of a homogeneous and glossy film with good hold on the nails.

The inventors have shown that it is possible to obtain such a composition by using phenylated silicone resins.

A subject-matter of the present invention is thus a nail varnish comprising an organic solvent medium, the said varnish comprising less than 5% of nitrocellulose and comprising at least one phenylated silicone resin.

In addition, the inventors have found that this resin makes it possible to significantly increase the gloss of the nail varnish compositions.

A further subject-matter of the invention is a cosmetic method for making up or for the nontherapeutic care of the nails comprising the application, to the nails, of at least one layer of a nail varnish as defined above.

Another subject-matter of the invention is the use of a nail varnish comprising an organic solvent medium and at least one phenylated silicone resin for obtaining, after deposition on the nail, a glossy film.

The nail varnish according to the invention comprises a cosmetically acceptable medium, that is to say a nontoxic medium capable of being applied to human keratinous substances, in particular the nails.

The nail varnish according to the invention comprises a liquid organic solvent medium and is provided in the form of a liquid or gel texture; it differs from a patch or from a soft article comprising a layer of polymeric material and a layer of adhesive intended to be adhesively bonded to the nail.

### Phenylated Silicone Resin

The nomenclature of silicone resins is known under the name of “MDTQ”, the resin being described as a function of the various monomeric siloxane units present in it, each of the letters “MDTQ” characterizing one type of unit.

The letter M represents the monofunctional unit of formula (R)₃SiO_{1/2}, the silicon atom being connected to just one oxygen atom in the polymer comprising this unit.

The letter D means a difunctional unit (R)₂SiO_{2/2} in which the silicon atom is connected to two oxygen atoms.

The letter T represents a trifunctional unit of formula (R)SiO_{3/2}.

In the units M, D and T defined above, at least one of the R groups can be an alkyl, aralkyl, alkaryl, aryl, carbinol or primary amine radical or else a hydroxyl group.

Finally, the letter Q means a tetrafunctional unit SiO_{4/2} in which the silicon atom is bonded to four oxygen atoms themselves bonded to the remainder of the polymer.

The phenylated silicone resin used in the composition according to the invention is advantageously an alkylphenylsilsesquioxane resin.

This resin advantageously comprises units of following formulae:

(R′SiO_{3/2})ₓ and (C₆H₅SiO_{3/2})_{y}
R′ being a linear or branched alkyl group comprising from 2 to 20 carbon atoms, preferably from 2 to 8 carbon atoms, a cycloalkyl group comprising from 5 to 20 carbon atoms, preferably from 6 to 12 carbon atoms, or a hydroxyl group, and
x and y independently ranging from 0 to 1, better still 0.05 to 0.95,
x and y representing the molar fraction of the siloxane units (R′SiO_{3/2})ₓ (T unit-alkylsiloxane) and (C₆H₅SiO_{3/2})_{y} (T unit-phenylsiloxane) with respect to the total number of moles of siloxane units in the phenylated silicone resin.

The phenylated silicone resin advantageously comprises at least 20 mol %, preferably at least 30 mol %, better still at least 40 mol %, even better still at least 50 mol % and better still at least 60 mol % of siloxane units (R′SiO_{3/2})ₓ and (C₆H₅SiO_{3/2})_{y}, with respect to the total number of siloxane units present in the phenylated silicone resin.

According to one embodiment, the content of siloxane units (R′SiO_{3/2})ₓ and (C₆H₅SiO_{3/2})_{y}, can range up to 100 mol %, with respect to the total number of siloxane units present in the phenylated silicone resin.

The phenylated silicone resin used in the composition according to the invention can additionally comprise M, D and Q units as defined above. These units, when they are present, are in a content preferably of less than or equal to 10 mol %, with respect to the total number of moles of siloxane units present in the phenylated silicone resin.

Use may thus be made of resins comprising units of following formula:

[R¹₃SiO_{1/2}]ₐ

[R²₂SiO_{2/2}]_{b}

[R³SiO_{3/2}]_{c}

[SiO_{4/2}]_{d}

[R′ SiO_{3/2}]ₓ and

[C₆H₅SiO_{3/2}]_{y}

in which:
R′ is an alkyl group comprising from 2 to 20 carbon atoms, preferably from 2 to 8 carbon atoms, or a cycloalkyl group comprising from 5 to 20 carbon atoms, preferably from 6 to 12 carbon atoms, or a hydroxyl group,
R¹, R² and R³ are chosen, independently of one another, from:
an alkyl group comprising from 1 to 20 carbon atoms, preferably from 1 to 8 carbon atoms,
a cycloalkyl group comprising from 5 to 20 carbon atoms, preferably from 6 to 12 carbon atoms,
an aralkyl group comprising from 7 to 14 carbon atoms,
an alkaryl group comprising from 7 to 14 carbon atoms,
an aryl group comprising from 6 to 10 carbon atoms, preferably 6 carbon atoms,
a carbinol group, or
a primary, secondary or tertiary amine group, and their combinations,
a, b, c and d independently range from 0 to 0.4, and
x and y independently range from 0 to 1, preferably from 0.05 to 0.95, with the condition that the sum x+y is greater than or equal to 0.2 and the sum a+b+c+d+x+y is equal to 1.

In particular, x can range from 0 to 0.95 and y can range from 0.05 to 1.

The term “carbinol group” is understood to mean a group comprising at least one carbon atom bonded to at least one OH group.

The carbinol group can thus be:
a noncyclic hydrocarbon chain comprising at least 3 carbon atoms, for example of formula R⁴OH, in which R⁴ is a hydrocarbon chain, optionally substituted by an oxygen atom, comprising at least 3 carbon atoms, such as, for example:
   CH₂CH(CH₃)—, —CH₂CH(CH₃)CH₂—,
   CH₂CH₂CH(CH₂CH₃)CH₂CH₂CH₂— and —OCH(CH₃)(CH₂)_{z}—, z ranging from 1 to 10, or
an aryl group comprising at least 6 carbon atoms, preferably from 6 to 14 carbon atoms, for example a group of formula R⁵OH, in which R⁵ is an arylene radical, such as, for example, —(CH₂)ₓC₆H₄—, with x ranging from 0 to 10, —CH₂CH(CH₃)(CH₂)ₓC₆H₄—, with x ranging from 0 to 10, or —(CH₂)ₓC₆H₄(CH₂)ₓ—, with x ranging from 1 to 10. The aryl carbinol group can comprise from 6 to 14 carbon atoms.

The primary, secondary or tertiary amine group can be chosen from the groups of formula —R⁶—NH₂ or —R⁶—NH—R⁷—, in which R⁶ is a divalent hydrocarbon radical comprising at least 2 carbon atoms, preferably from 2 to 20 carbon atoms, and R⁷ represents ethylene, propylene, —CH₂CHCH₃—, butylene, —CH₂CH(CH₃)CH₂—, pentamethylene, hexamethylene, 3-ethylhexamethylene, octamethylene, decamethylene and their mixtures.

Preferably, R′ is a linear or branched alkyl group comprising from 2 to 8 carbon atoms, such as methyl, ethyl, propyl, butyl, pentyl, hexyl and octyl, and more preferably a propyl group (the resin is then a propylphenylsilsesquioxane resin).

Advantageously, the R¹, R² and R³ radicals are chosen, independently of one another, from alkyl groups, such as methyl, ethyl, propyl, butyl, pentyl, hexyl and octyl.

According to one embodiment, R¹ and R³ are methyl groups and R² is a methyl or phenyl group.

According to one embodiment, the number of methylsilsesquioxane units is less than or equal to 30% as number of moles, with respect to the total number of moles of siloxane units present in the phenylated silicone resin, preferably less than or equal to 20 mol %.

According to one embodiment, the units (R′SiO_{3/2})ₓ and (C₆H₅SiO_{3/2})_{y} are preferably present in a molar ratio such that the number of phenyl groups to the number of C₂-C₂₀ alkyl groups ranges from 1/10 to 10/1.

Such resins are described in particular in the documents WO2005/090444 and US 2004/0180011, the contents of which are incorporated by reference in the present patent application.

According to a specific embodiment, the composition according to the invention comprises at least 2 phenylated silicone resins, preferably two alkylphenylsilsesquioxane resins, which correspond in particular to the formulae defined above.

In particular, the composition comprises:
at least one resin (i) comprising units of following formulae:

(R′SiO_{3/2})_{x′ and (C}₆H₅SiO_{3/2})_{y′}
R′ being as defined above,
x′ ranging from 0 to 0.3, for example from 0.05 to 0.3, and
y′ ranging from 0.7 to 1, and
at least one resin (ii) comprising units of following formulae:

(R′SiO_{3/2})_{x″} and (C₆H₅SiO_{3/2})_{y″}
R′ being as defined above,
x″ ranging from 0.3 to 1, and
y″ ranging from 0 to 0.7.

The phenylated silicone resin or resins can be present in a content, as active material, ranging from 0.1% to 60% by weight, with respect to the total weight of the composition, preferably ranging from 2% to 50% by weight, better still from 5% to 45% by weight and better still from 5% to 40% by weight.

Mention may be made, as commercial references for phenylated silicone resins which can be used in the present invention, to the resins sold by Dow Corning under the references Z-6018 Intermediate or Z-217 Flake resin or by Wacker under the reference Belsil SPR 45 VP.

### Organic Solvent Medium

The nail varnish composition according to the invention comprises an organic solvent medium comprising one or more nonaqueous compounds which are liquid at ambient temperature, also known as oils or organic solvents.

The organic solvent can be chosen from:
ketones which are liquid at ambient temperature, such as methyl ethyl ketone, methyl isobutyl ketone, diisobutyl ketone, isophorone, cyclohexanone or acetone;
alcohols which are liquid at ambient temperature, such as ethanol, isopropanol, diacetone alcohol, 2-butoxyethanol or cyclohexanol;
propylene glycol ethers which are liquid at ambient temperature, such as propylene glycol monomethyl ether, propylene glycol monomethyl ether acetate or dipropylene glycol mono(n-butyl) ether;
cyclic ethers, such as γ-butyrolactone;
short chain esters (having from 3 to 8 carbon atoms in total), such as ethyl acetate, methyl acetate, propyl acetate, isopropyl acetate, n-butyl acetate, isopentyl acetate, methoxypropyl acetate, t-butyl acetate or butyl lactate;
ethers which are liquid at ambient temperature, such as diethyl ether, dimethyl ether or dichlorodiethyl ether;
alkanes which are liquid at ambient temperature, such as decane, heptane, dodecane or cyclohexane;
alkyl sulphoxides, such as dimethyl sulphoxide;
aldehydes which are liquid at ambient temperature, such as benzaldehyde or acetaldehyde;
ethyl 3-ethoxypropionate;
carbonates, such as propylene carbonate or dimethyl carbonate;
acetals, such as methylal;
and their mixtures.

The organic medium can comprise at least one volatile or nonvolatile silicone oil.

The term “volatile oil” is understood to mean, within the meaning of the invention, an oil capable of evaporating on contact with keratinous substances in less than one hour at ambient temperature and atmospheric pressure. The volatile organic solvent or solvents and the volatile oils of the invention are volatile cosmetic organic solvents and oils which are liquid at ambient temperature and which have a nonzero vapour pressure, at ambient temperature and atmospheric pressure, ranging in particular from 0.13 Pa to 40,000 Pa (10⁻³ to 300 mmHg), in particular ranging from 1.3 Pa to 13,000 Pa (0.01 to 100 mmHg) and more particularly ranging from 1.3 Pa to 1300 Pa (0.01 to 10 mmHg).

The term “nonvolatile oil” is understood to mean an oil which remains on keratinous substances at ambient temperature and atmospheric pressure for at least several hours and which has in particular a vapour pressure of less than 10⁻³ mmHg (0.13 Pa).

The volatile silicone oils can be linear or cyclic volatile silicone oils, in particular those having a viscosity≦8 centistokes (8×10⁻⁶ m²/s) and having in particular from 2 to 7 silicon atoms, these silicones optionally comprising alkyl or alkoxy groups having from 1 to 10 carbon atoms. Mention may in particular be made, as volatile silicone oils which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Mention may also be made of the volatile linear alkyltrisiloxane oils of general formula (I)

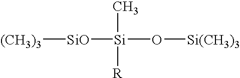

where R represents an alkyl group comprising from 2 to 4 carbon atoms, one or more hydrogen atoms of which can be substituted by a fluorine or chlorine atom.

Mention may be made, among the oils of general formula (I), of:

3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane, corresponding to the oils of formula (I) for which R is respectively a butyl group, a propyl group or an ethyl group.

The nonvolatile silicone oils which can be used in the composition according to the invention can be polydimethylsiloxanes (PDMSs) which are nonvolatile, polydimethylsiloxanes comprising pendant alkyl or alkoxy groups and/or alkyl or alkoxy groups at the end of the silicone chain, groups each having from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Use may also be made of fluorosilicone oils, such as, for example, those which are described in the document EP-A-847 752.

Preferably, the solvent is chosen from short chain esters having from 3 to 8 carbon atoms in total, such as ethyl acetate, methyl acetate, propyl acetate, isopropyl acetate, n-butyl acetate, isopentyl acetate, methoxypropyl acetate, butyl lactate and their mixtures.

The organic solvent medium can represent from 10 to 95% by weight, with respect to the total weight of the composition, preferably from 15 to 80% by weight and better still from 20 to 70% by weight.

### Aqueous Medium

The composition according to the invention can also comprise an aqueous medium.

The content of aqueous medium in the composition can range from 5 to 95% by weight, with respect to the total weight of the composition, preferably from 30 to 70% by weight.

Preferably, the composition comprises less than 10% by weight of water, preferably less than 5% by weight, with respect to the total weight of the composition.

### Additional Film-forming Polymer

The nail varnish composition comprises less than 5% by weight, as dry matter, of nitrocellulose, with respect to the total weight of the composition, preferably less than 3% by weight, preferably less than 1.5% by weight, preferably less than 1% by weight. In particular, the composition is devoid of nitrocellulose.

The composition according to the invention can comprise, in addition to the phenylated silicone resin, an “additional” film-forming polymer.

The term “film-forming polymer” is understood to mean, within the meaning of the present invention, a polymer capable of forming, by itself alone or in the presence of a subsidiary film-forming agent, a continuous film on a substrate, in particular on keratinous substances.

Mention may be made, among additional film-forming polymers which can be used in the composition of the present invention, of synthetic polymers, of radical type or of polycondensate type, polymers of natural origin and their blends.

The additional film-forming polymer can be chosen in particular from cellulose polymers, such as cellulose acetate, cellulose acetate butyrate, cellulose acetate propionate or ethyl cellulose, or alternatively polyurethanes, acrylic polymers, vinyl polymers, polyvinylbutyrals, alkyd resins, ketone/aldehyde resins, resins resulting from aldehyde condensation products, such as arylsulphonamide/formaldehyde resins, for example toluenesulphonamide/formaldehyde resin, arylsulphonamide/epoxy resins or ethyl tosylamide resins.

Use may in particular be made, as additional film-forming polymer, of the toluenesulphonamide/formaldehyde resins “Ketjentflex MS80” from Akzo or “Santolite MHP” or “Santolite MS 80” from Faconnier or “Resimpol 80” from Pan Americana, the alkyd resin “Beckosol ODE 230-70-E” from Dainippon, the acrylic resin “Acryloid B66” from Röhm & Haas, the polyurethane resin “Trixene PR 4127” from Baxenden or the acetophenone/formaldehyde resin sold under the reference Synthetic Resin SK by Degussa.

The film-forming polymer can be a film-forming silicone polymer chosen from:
the silicone resins, other than the phenylated silicone resin, comprising units M, D, T and Q as defined above, such as siloxysilicate resins. Mention may be made of trimethylsiloxysilicate (TMS) resins, such as that sold under the reference SR1000 by General Electric or under the reference TMS 803 by Wacker. Mention may also be made of trimethylsiloxysilicate resins marketed in a solvent, such as cyclomethicone, sold under the name “KF-7312J” by Shin-Etsu and “DC 749” and “DC 593” by Dow Corning,
acrylic and vinyl copolymers grafted with silicone or acrylic/silicone block copolymers comprising at least 3 blocks, such as:
   a) fat-soluble acrylic polymers grafted with silicones, such as, for example, the SA 70 polymers from 3M, alkyl methacrylate copolymers grafted with polydimethylsiloxane, such as KP 545 and KP 550 from Shin Etsu, or silicone polymers grafted with acrylics, such as, for example, VS 70 and VS 80 from 3M,
   b) vinyl polymers having at least one unit derived from a carbosiloxane dendrimer, preferably in the form of grafts; such polymers are described, for example, in the documents EP 0 963 751 or WO 03/045337, the contents of which are incorporated by reference in the present patent application,
   c) dispersions of particles of acrylic and vinyl polymers in an oil or an organic solvent and in particular:
      particles of acrylic polymers, in dispersion in a liquid fatty phase and stabilized at the surface by a stabilizing agent, chosen from silicone polymers grafted with a hydrocarbon chain, such as described in the document EP 0 749 746,
      particles of polymers formed of an ethylenic polymer grafted with silicone, preferably an acrylic polymer grafted with silicone, dispersed in a liquid fatty phase, the ethylenic polymer advantageously being dispersed in the absence of an additional stabilizing agent at the surface of the particles, such as described in particular in the document EP 1 428 843,
   d) dispersions in water of particles of acrylic/silicone hybrid polymers with crosslinking between the polyacrylates and the silicone, such as those produced by Dainippon Inc & Chemical and described in the document “Aqueous dispersion of polysiloxane/acrylic hybrid resins for coatings”, Kudo, in Chemical and Polymer silicones in coatings—Conf. Barcelona, paper 31, pages 1-8,
polycondensates/silicones, such as:
   a) silicone/polyurethane, silicone/polyurethane/urea, silicone/polyurea polyurethane or polyurethane/urea polycondensates and polyurea polymers comprising silicone blocks, such as, for example:
      aqueous dispersions (or latexes) of polyurethane/silicone multiblock polymers carrying ionic or ionisable groups, such as described, for example, in the documents EP 0 751 162 and EP 0 782 881,
      silicone/polyurethane block elastomers, preferably soluble in ethanol or in another polar solvent, such as, for example, those described in the document WO 2003/014194 and sold by Wacker under the reference Geniomer, and their mixtures,
         b) silicone polyamides of polyorganosiloxane type, such as, for example, those described in the documents U.S. Pat. No. 5,874,069, U.S. Pat. No. 5,919 441, U.S. Pat. No. 6,051,216 and U.S. Pat. No. 5,981,680. Mention may be made, for example, of the polyamide/polydimethylsiloxane sold under the reference DC 2-8179 by Dow Corning,
crosslinked silicone polymers and organic/silicone hybrid polymers (crosslinked or noncrosslinked) obtained by sol-gel technology.

Polymers obtained by such a process are described, for example, in Application WO 98/44906, the content of which is incorporated by reference in the present patent application.

These polymers can be vinyl/silicone or acrylic/silicone hybrid polymers, or silicone polycondensates.

According to one embodiment of the invention, the additional film-forming polymer is a film-forming ethylenic linear block polymer which preferably comprises at least one first block and at least one second block having different glass transition temperatures (Tg), the said first and second blocks being connected to one another via an intermediate block comprising at least one constituent monomer of the first block and at least one constituent monomer of the second block.

Advantageously, the first and second blocks of the block polymer are incompatible with one another.

Such polymers are described, for example, in the documents EP 1 411 069 or WO 04/028488.

The additional film-forming polymer can be present in the composition according to the invention in a content ranging from 0.1% to 30% by weight, with respect to the total weight of the composition, preferably ranging from 0.5% to 20% by weight and better still from 1% to 10% by weight.

### Subsidiary Film-forming Agent

A subsidiary film-forming agent may be provided in order to improve the film-forming properties of the nail varnish composition.

Such a subsidiary film-forming agent can be chosen from any compound known to a person skilled in the art as being capable of fulfilling the desired purpose and can in particular be chosen from plasticizing agents and coalescence agents for the film-forming polymer or polymers.

Thus, the composition can additionally comprise at least one plasticizing agent and/or one coalescence agent. Mention may in particular be made, alone or as a mixture, of the usual plasticizers and coalescence agents, such as:
glycols and their derivatives, such as diethylene glycol ethyl ether, diethylene glycol methyl ether, diethylene glycol butyl ether or diethylene glycol hexyl ether, ethylene glycol ethyl ether, ethylene glycol butyl ether or ethylene glycol hexyl ether;
glycol esters;
propylene glycol derivatives and in particular propylene glycol phenyl ether, propylene glycol diacetate, dipropylene glycol ethyl ether, tripropylene glycol methyl ether and diethylene glycol methyl ether, or propylene glycol butyl ether;
acid esters, in particular carboxylic acid esters, such as citrates, phthalates, adipates, carbonates, tartrates, phosphates or sebacates;
oxyethylenated derivatives, such as oxyethylenated oils, in particular vegetable oils, such as castor oil; and
their mixtures.

Some silicone oils mentioned above can act as coalescence agent or plasticizing agent.

The type and the amount of plasticizing agent and/or coalescence agent can be chosen by a person skilled in the art on the basis of his general knowledge.

For example, the content of plasticizing agent and/or coalescence agent agent can range from 0.01% to 20% by weight and in particular from 0.5% to 10% by weight, with respect to the total weight of the composition.

### Gelling Agent

The composition can comprise a gelling agent.

This gelling agent can in particular be chosen from: hydrophobic silicas, such as those described in the document EP-A-898 960, for example sold under the references “Aerosil R812®” by Degussa, “Cab-O-Sil TS-530®”, “Cab-O-Sil TS-610®” and “Cab-O-Sil TS-720®” by Cabot and “Aerosil R972®” and “Aerosil R974®” by Degussa, clays, such as montmorillonite, modified clays, such as bentones, for example stearalkonium hectorite or stearalkonium bentonite, or polysaccharide alkyl ethers (in particular for which the alkyl group comprises from 1 to 24 carbon atoms, preferably from 1 to 10, better still from 1 to 6 and more especially from 1 to 3), such as those described in the document EP-A-898 958.

The total proportion of thickening agent(s) in the compositions according to the invention can range from 0.01 to 15% by weight, with respect to the total weight of the composition, preferably from 0.5 to 15% by weight and better still from 0.5 to 10% by weight.

### Colouring Material

The composition according to the invention can additionally comprise one or more colouring materials chosen from water-soluble dyes and pulverulent colouring materials, such as pigments, pearlescent agents and glitters well known to a person skilled in the art. The colouring materials can be present in the composition in a content ranging from 0.01% to 50% by weight, with respect to the weight of the composition, preferably from 0.01% to 30% by weight.

The term “pigments” should be understood as meaning white or coloured and inorganic or organic particles of any shape which are insoluble in the physiological medium and which are intended to colour the composition.

The term “pearlescent agents” should be understood as meaning iridescent particles of any shape, in particular produced by certain molluscs in their shells or else synthesized.

The pigments can be white or coloured and inorganic and/or organic. Mention may be made, among inorganic pigments, of titanium dioxide, optionally surface treated, zirconium or cerium oxides, zinc, iron or chromium oxides (the iron oxides being black, yellow or red), manganese violet, ultramarine blue, chromium hydrate, ferric blue or metal powders, such as aluminium powder or copper powder.

Mention may be made, among organic pigments, of carbon black, pigments of D & C type, and lakes based on cochineal carmine of barium, strontium, calcium or aluminium.

The pearlescent pigments can be chosen from white pearlescent pigments, such as mica covered with titanium oxide or with bismuth oxychloride, coloured pearlescent pigments, such as titanium oxide-coated mica covered with iron oxides, titanium oxide-coated mica covered with in particular ferric blue or with chromium oxide, or titanium oxide-coated mica covered with an organic pigment of the abovementioned type, and pearlescent pigments based on bismuth oxychloride.

The water-soluble dyes are, for example, beetroot juice or methylene blue.

The composition according to the invention can additionally comprise one or more fillers, in particular in a content ranging from 0.01% to 50% by weight, with respect to the total weight of the composition, preferably ranging from 0.01% to 30% by weight. The term “fillers” should be understood as meaning colourless or white and inorganic or synthetic particles of any shape which are insoluble in the medium of the composition, whatever the temperature at which the composition is manufactured. These fillers are used in particular to modify the rheology or the texture of the composition.

The fillers can be inorganic or organic and of any shape, platelet, spherical or oblong, whatever the crystallographic form (for example sheet, cubic, hexagonal, orthorhombic, and the like). Mention may be made of talc, mica, silica, kaolin, polyamide (Nylon®) powders (Orgasol® from Atochem), poly-β-alanine powders, polyethylene powders, powders formed of tetrafluoroethylene polymers (Teflon®), lauroyllysine, starch, boron nitride, polymeric hollow microspheres, such as those of polyvinylidene/acrylonitrile chloride, for example Expancel® (Nobel Industrie), or of acrylic acid copolymers (Polytrap® from Dow Corning), silicone resin microbeads (Tospearls® from Toshiba, for example), polyorganosiloxane elastomer particles, precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

### OTHER ADDITIVES

The composition can additionally comprise other ingredients commonly used in cosmetic compositions. Such ingredients can be chosen from spreading agents, wetting agents, dispersing agents, antifoaming agents, preservatives, UV screening agents, active principles, surfactants, moisturizing agents, fragrances, neutralizing agents, stabilizing agents or anti-oxidants.

Of course, a person skilled in the art will take care to choose this or these optional additional compounds and/or their amounts so that the advantageous properties of the composition for the use according to the invention are not, or not substantially, detrimentally affected by the envisaged addition.

According to another aspect, a subject-matter of the invention is a nail varnish product comprising: i) a container delimiting at least one compartment, the said container being closed by a closing element, and ii) a composition according to the invention which is positioned inside the said compartment.

The container can have any appropriate form. It can in particular be in the form of a bottle and can, at least in part, be made of a material such as glass. However, materials other than glass can be used, such as thermoplastics, for example PP or PE, or such as a metal.

The closing element can be coupled to the compartment by screwing into the closed position of the container.

Alternatively, the coupling between the closing element and the container can be carried out other than by screwing, in particular by snapping.

The container is preferably equipped with an applicator which can be in the form of a brush composed of at least one tuft of hairs. Alternatively, the applicator is provided in a form other than a brush, for example in the form of a spatula or of a foam tip.

The invention is illustrated in more detail in the following examples. Unless otherwise indicated, the amounts are given as percentage by weight with respect to the total weight of the composition.

### EXAMPLE 1

### Colourless Nail Varnish

The following nail varnish was prepared:

| | |
|---|---|
| Hectorite modified with | 3.57 |
| stearylbenzyldimethylammonium | |
| (Bentone 27 V from Elementis) | |
| Polyphenylsiloxane resin | 50 |
| (Dow Corning Z-6018 Intermediate) | |
| Isopropyl alcohol | 1.28 |
| Tributyl acetylcitrate | 3 |
| Butyl acetate | 12 |
| Ethyl acetate | q.s. for 100 |
| Citric acid monohydrate | 0.14 |

### EXAMPLE 2

### Colourless Nail Varnish

The following nail varnish, which comprises two phenylated silicone resins, was prepared:

| | |
|---|---|
| Hectorite modified with | 3.57 |
| stearylbenzyldimethylammonium | |
| (Bentone 27 V from Elementis) | |
| Phenylpolysilsesquioxane resin | 20 |
| (Belsil SPR 45 VP from Wacker) | |
| Phenyl silicone resin | 20 |
| (Dow Corning Z-217 Flake resin) | |
| Isopropyl alcohol | 1.28 |
| Tributyl acetylcitrate | 3 |
| Butyl acetate | 12 |
| Ethyl acetate | q.s. for 100 |
| Citric acid monohydrate | 0.14 |

### EXAMPLE 3

### Coloured Nail Varnish

The following nail varnish was prepared:

| | |
|---|---|
| Hectorite modified with | 3.48 |
| stearylbenzyldimethylammonium | |
| (Bentone 27 V from Elementis) | |
| Phenylpolysilsesquioxane resin | 19.5 |
| (Belsil SPR 45 VP from Wacker) | |
| Phenyl silicone resin | 19.5 |
| (Dow Corning Z-217 Flake resin) | |
| Isopropyl alcohol | 1.25 |
| Tributyl acetylcitrate | 2.92 |
| Pigments and pearlescent agents | 2.46 |
| Butyl acetate | 11.7 |
| Ethyl acetate | q.s. for 100 |
| Citric acid monohydrate | 0.14 |

1. A nail varnish comprising an organic solvent medium, wherein
the organic solvent medium comprises less than 1.5% of nitrocellulose and at least one phenylated silicone resin.

2. The nail varnish according to claim 1, wherein the phenylated silicone resin is an alkylphenylsilsesquioxane resin.

3. The nail varnish according to claim 1, wherein
the silicone resin comprises units represented by
(R′SiO_{3/2})ₓ and (C₆H₅SiO_{3/2})_{y};
wherein
R′ is a linear or branched alkyl group comprising from 2 to 20 carbon atoms, a cycloalkyl group comprising from 5 to 20 carbon atoms or a hydroxyl group, and
x and y independently of one another are in the range from 0 to 1.

4. The nail varnish according to claim wherein
the phenylated silicone comprises at least 20 mol % of siloxane units (R′SiO_{3/2})ₓ and (C₆H₅SiO_{3/2})_{y}, with respect to the total number of siloxane units present in the phenylated silicone resin.

5. The nail varnish according to claim 1, wherein
the phenylated silicone comprises units represented by:
[R¹₃SiO_{1/2}]ₐ
[R²₂SiO_{2/2}]_{b}
[R³SiO_{3/2}]_{c}
[SiO_{4/2}]_{d}
[R′SiO_{3/2}]ₓ and
[C₆H₅SiO_{3/2}]_{y}
wherein:
R′ is an alkyl group comprising from 2 to 20 carbon atoms or a cycloalkyl group comprising from 5 to 20 carbon atoms or a hydroxyl group,
R¹, R² and R³ are selected, independently of one another, from the group consisting of:
an alkyl group comprising from 1 to 20 carbon atoms,
a cycloalkyl group comprising from 5 to 20 carbon atoms,
an aralkyl group comprising from 7 to 14 carbon atoms,
an alkaryl group comprising from 7 to 14 carbon atoms,
an aryl group comprising from 6 to 10 carbon atoms,
a carbinol group,
a primary, secondary or tertiary amine group, and their combinations,
a, b, c and d independently range from 0 to 0.4, and
x and y independently range from 0 to 1,
wherein
the sum x+y is greater than or equal to 0.2 and
the sum a+b+c+d+x+y is equal to 1.

6. The nail varnish according to claim 3 wherein R′ is a linear or branched alkyl group comprising from 2 to 8 carbon atoms.

7. The nail varnish according to claim 3 wherein R′ is a propyl group.

8. The nail varnish according to claim 5 wherein the R¹, R² and R³ radicals are selected, independently of one another, from the group of alkyl groups, consisting of methyl, ethyl, propyl, butyl, pentyl, hexyl and octyl.

9. The nail varnish according to claim 5 wherein the R¹ and R³ radicals are methyl groups and R² is a methyl or phenyl group.

10. The nail varnish according to claim 3 wherein x and y independently range from 0.05 to 0.95.

11. The nail varnish according to claim 1, wherein the phenylated silicone resin is a propylphenylsilsesquioxane resin.

12. The nail varnish according to claim 1, which comprises at least two phenylated silicone resins.

13. The nail varnish according to claim 12, wherein the at least two phenylated silicone resins are at least two alkylphenylsilsesquioxane resins.

14. The nail varnish according to claim 1 which comprises:
at least one resin (i) comprising units represented by
(R′SiO_{3/2})ₓ, and (C₆H₅SiO_{3/2})_{y},
wherein
R′ is defined above,
x′ is from 0 to 0.3, and
y′ is from 0.7 to 1; and
at least one resin (ii) comprising units represented by:
(R′SiO_{3/2})_{x″} and (C₆H₅SiO_{3/2})_{y″}
wherein
R′ is defined above,
x″ is from 0.3 to 1, and
y″ is from 0 to 0.7.

15. The nail varnish according to claim 1, wherein a % by weight of the phenylated silicone resin or resins is from 0.1% to 60% by weight, with respect to the total weight of the composition.

16. The nail varnish according to claim 1, wherein the organic solvent medium comprises at least one organic solvent which is a short chain ester having from 3 to 8 carbon atoms in total.

17. The nail varnish according to claim 1, wherein a % by weight of the organic solvent medium is from 10 to 95% by weight, with respect to the total weight of the composition.

18. The nail varnish according to claim 1, which comprises no nitrocellulose.

19. The nail varnish according to claim 1, which further comprises at least one gelling agent.

20. The nail varnish according to claim 19, wherein a % by weight of the gelling agent is from 0.01 to 15% by weight, with respect to the total weight of the composition.

21. The nail varnish according to claim 1, which further comprises at least one colouring material.

22. The nail varnish according to claim 21, wherein a % by weight of the colouring material is from 0.01% to 50% by weight, with respect to the weight of the composition.

23. A cosmetic method for making up or for a nontherapeutic care of nails, comprising, applying to the nails, at least one layer of the nail varnish according to claim 1.

24. The cosmetic method according to claim 23, wherein the at least one layer of the nail varnish is a glossy film.
